(19) United States
(12) Patent Application Publication
(10) Pub. No.: US 2004/0016892 A1
(43) Pub. Date: Jan. 29, 2004

(54) Method and system for electronic pasteurization

(76) Inventors: Peter M. McIntyre, College Station, TX (US); Erwin M. Thomas III, Bryan, TX (US)

Correspondence Address: MUNSCH, HARDT, KOPF & HARR, P.C., INTELLECTUAL PROPERTY DOCKET CLERK, 1445 ROSS AVENUE, SUITE 4000, DALLAS, TX, 75202-2790, US

(21) Appl. No.: 10/293,202

(22) Filed: Nov. 13, 2002

Related U.S. Application Data
(60) Provisional application No. 60/338,191, filed on Nov. 13, 2001.

(51) Int. Cl. A61L 2/08; G21K 5/10

(52) U.S. Cl. 250/492.3; 250/455.11

(57) ABSTRACT

An improved electronic pasteurization method and system is presented. The improved electronic pasteurization system includes a coupled accelerator and a treatment station. The coupled accelerator includes a coupled multiplier supply (CMS) having a mechanical drive system and power modules. The mechanical drive system supplies mechanical power to the power modules, which convert the mechanical power into electrical power that provides stepped-up power to the accelerator column.

### CO-PENDING APPLICATIONS

This application claims priority under 35 U.S.C. § 119(e) to U.S. Provisional Application No. 60/338,191, entitled Coupled Multiplier Supply And Coupled Electronic Pasteurization System, having a filing date of Nov. 13, 2001. This application is related to co-pending U.S. patent application Ser. No. 09/248,675, entitled Method and System for Electronic Pasteurization, having a filing date of Feb. 11, 1999.

### FIELD OF THE DISCLOSURE

This invention relates in general to the field of high voltage electronic systems, and more particularly to a coupled multiplier supply and coupled electronic pasteurization system.

### BACKGROUND

Irradiation involves exposing a target to an ionizing radiation to change the microbiology of the target. Irradiation is an effective method for killing microorganisms and insects in foods, extending the shelf life of various foods, and sterilizing medical products. Irradiation is particularly suited for treating food, such as meat, to kill food-borne pathogens, such as E. Coli, Trichinosis, Salmonella, Yersinia, Campylobacter, Shigella, and the like. Two characteristics determine the effectiveness of an irradiation treatment—the dose, which is the total beam energy delivered per mass of food; and the penetration depth, which is the maximum depth into the food to which the dose is delivered. The penetration depth is a property of the ionizing radiation that is used for irradiation. Food irradiation typically requires a dose of about 300,000 rads to achieve a statistical kill of the pathogenic bacteria.

Conventional irradiation systems utilize one of three methods to produce the ionizing radiation. Gamma-ray irradiation systems produce y-rays from radioactive sources, typically Co⁶⁰. X-ray irradiation systems produce X-rays by targeting an electron beam, generally on the order of 5 MV (mega electron volts), onto a metal target. Conventional electron beam irradiation systems produce a high-energy electron beam, typically on the order of 10 MV energy, and deliver it directly into the food. The γ-ray irradiation systems and X-ray irradiation systems deliver a deep penetration, on the order of 30 centimeters into food, but require immense shielding assemblies, on the order of three meters thickness of concrete, for safe operation. Conventional electron beam irradiation systems deliver less penetration, typically less than seven centimeters in food, but require somewhat less shielding, on the order of two meters of concrete.

Conventional electron beam irradiation systems generally include an accelerator, a beam transport system, and a treatment station. Specifically, the accelerator produces an electron beam which is communicated to the treatment station by the beam transport system. Within the treatment station, the electron beam is scanned to deliver a uniform dose as the target passes through the treatment station. The higher the energy of the electron beam, the greater the depth that the electron beam can penetrate the target and deliver the required dose of ionizing radiation.

Conventional electron beam irradiation systems have many disadvantages. For example, conventional electron beam irradiation systems are inefficient, in that the electron beam scans across a specific area within the treatment station, but in many applications, the target covers only a fraction of the scanned area. The utilization efficiency, defined as the fraction of electron beam actually delivered to target, is typically less than 30% in conventional electron beam irradiation systems.

As will be discussed below, conventional electron beam irradiation systems are extremely expensive due to the technical disadvantages of conventional accelerators, beam transport systems, and treatment stations. Conventional electron beam irradiation systems often utilize a radio-frequency linear accelerator (LINAC) to produce an electron beam. LINACs operate by producing a high-intensity electric field within a series of cylindrically symmetric resonant cavities. The electron beam is passed along the axis of the cavities, where it is both accelerated, to increase its energy, and focused, to confine the beam transversely.

One technical disadvantage of LINACs is that only a single electron beam can be produced, because the beam must pass along the axis of the cavities. For food irradiation applications, a typical food processing operation has multiple parallel processing lines, and thus requires multiple parallel treatment stations for irradiation to avoid product “choke points.” However, because a conventional electron beam irradiation system can produce only one beam for irradiation, another machine (accelerator) would be required in order to have more than one beam, thereby making multiple irradiation stations very expensive.

Further disadvantages of conventional accelerators are their expense to construct, their limited beam power, and their operating efficiency. Conventional industrial irradiation accelerators, e.g., 5-10 MV energy, can cost on the order of five million dollars, or more, and these accelerators usually operate at only 30-70 percent efficiency.

Conventional beam transport systems generally utilize electromagnets to transport the electron beam from the accelerator to the treatment station. The electromagnets generate a magnetic field based on the pattern of electrical currents that flow through the electromagnet. Conventional beam transport systems generally use dipole and quadrupole electromagnets. The dipole electromagnet produces a uniform magnetic field in the region traversed by the beam and thereby bends the electron beam on a constant radius of curvature. The quadrupole electromagnet produces a distribution of magnetic field that increases linearly with distance from the beam axis, and focuses the beam to confine it along the direction of transport. A technical disadvantage of conventional beam transport systems is that the electromagnets require an active electrical power system along the entire length of the beam transport system. The electrical power system adds complexity and cost to conventional beam transport systems, particularly in food irradiation applications, where it may be advantageous to locate the accelerator in one location and deliver beams to multiple treatment stations at locations distributed throughout a large facility.

Conventional treatment stations include electro-optics that scan the electron beam transversely to illuminate the scan area, as well as shielding to prevent harmful levels of radiation from escaping the treatment station. Conventional electro-optics direct the electron beam to the outer surfaces of the target. A technical disadvantage of conventional electro-optics is that the internal cavities cannot readily be irradiated with the electron beam unless the beam energy is sufficiently high to penetrate the entire thickness of the target. However, higher-energy electron beams necessitate the use of greater shielding thickness to protect operating personnel from exceeding radiation protection dose limits.

Conventional irradiation systems must be housed in a structure that shields the intense ionizing radiation to permit operations in a food processing plant without exceeding the radiation protection dose limits either to radiation workers, or to members of the general public. Federal and state regulatory agencies require the dose from man-made ionizing radiation sources be as low as reasonably achievable (ALARA) for trained radiation workers, in order to avoid genetic/somatic effects and cancer risk. The current whole-body occupational radiation exposure limit from external exposure to man-made ionizing radiation sources is 5,000 millirem (mrem). In addition, the current regulatory whole-body dose limit to members of the public, excluding medical procedures, must not exceed 100 mrem per year from man-made sources of ionizing radiation. This lower dose value is a level commensurate with the radiation dose a person would naturally receive from “background” radiation sources, e.g., cosmic rays and naturally occurring radioactive materials in the earth, air, and water, over the course of one year. For example, in New York City, the average background radiation dose is approximately 88 mrem, while a Denver, Colo. resident's annual background radiation dose would be closer to 300 mrem.

In order to reduce the dose of ionizing radiation to acceptable regulatory/ALARA levels, the thickness of the shielding in conventional irradiation systems of 5-10 MV beam energy typically exceeds three meters, and is often on the order of five meters. In addition, the shielding must include a labyrinth or maze having a similar thickness through which the target is transported in and out of the treatment station. A typical conventional irradiation system occupies an area of about 200 m², which makes it difficult to integrate into the existing process lines of a food processing plant.

A disadvantage of large shielding thicknesses is that the treatment station often requires a separate shielded facility that cannot be easily integrated into existing large, in-line food processing applications. Another disadvantage is that the separate shielded facility creates a processing bottleneck or choke point, in that all targets must pass through this one facility. Furthermore, the capital costs associated with constructing the shielding facility often approach or exceed the cost of the accelerator.

Even though the Federal government has permitted the irradiation of food products for some time now, because of construction costs, operating expenses, and radiation safety issues associated with conventional industrial irradiation systems, these systems have not generally been commercially implemented on a large scale for food treatment purposes.

### SUMMARY OF THE INVENTION

One implementation of the present invention comprises an improved pasteurization system. In one embodiment, the improved pasteurization system comprises a coupled accelerator and a treatment station. In this embodiment, the coupled accelerator comprises an electron gun, one or more power modules coupled to a mechanical drive system, and at least one accelerator column. The mechanical drive system supplies mechanical power to the power modules, which converts the mechanical power to electrical power that is supplied, in part, to the at least one accelerator column. The coupled accelerator produces at least one electron beam that is delivered to the treatment station where a target is irradiated with the electron beam.

The present invention provides certain technical advantages over conventional systems and methods. Particular implementation and embodiments of the present invention may have all, some, or none of these technical advantages. For example, in some embodiments, the power modules and drive system enable the power of the coupled accelerator to be scaled without incurring substantial costs and complexity.

Other advantages will be readily apparent to one skilled in the art from the following figures, descriptions, and claims.

### BRIEF DESCRIPTION OF THE DRAWINGS

For a more complete understanding of the present invention and for further features and advantages thereof, reference is now made to the following description taken in conjunction with the accompanying drawings, wherein like reference numerals represent like features, in which:

FIG. 1 is a simplified block diagram of a coupled electronic pasteurization system in accordance with the present invention;

FIG. 2 is a simplified schematic diagram of an accelerator in accordance with the present invention;

FIG. 3 is an electrical schematic diagram, in part, of a power module in accordance with the present invention;

FIG. 4 is a schematic diagram of an alternator in accordance with the present invention;

FIGS. 5a-5d illustrate a circuit diagram of the control circuit for one power module in accordance with the present invention;

FIG. 6 is a circuit diagram of a current monitor circuit in accordance with the present invention;

FIG. 7 is a circuit diagram of a shunt circuit in accordance with the present invention;

FIG. 8 is a model picture of an isolation hub in accordance with the present invention;

FIG. 9 is a perspective cutaway view, in part, of the coupled multiplier supply integrated with two electron accelerators as load for a coupled pasteurization system in accordance with the present invention;

FIG. 10 is a simplified diagram showing a waste stream containing a chemical compound being treated according to at least one embodiment of the present invention; and

FIGS. 11A-11C are graphs showing voltage waveforms showing the response of a power module under full load current (100 mA) according to at least one embodiment of the present invention.

### DETAILED DESCRIPTION OF THE FIGURES

This application discloses a number of inventions relating to an improved electronic pasteurization system. This application describes a number of inventions. It should be understood that the individual inventions may be used individually, in any combination, or in total, depending upon the implementation. In addition, the present inventions may also be used in other electronic apparatus not directly relating to electronic pasteurization or electron beam accelerators without departing from the scope of the invention.

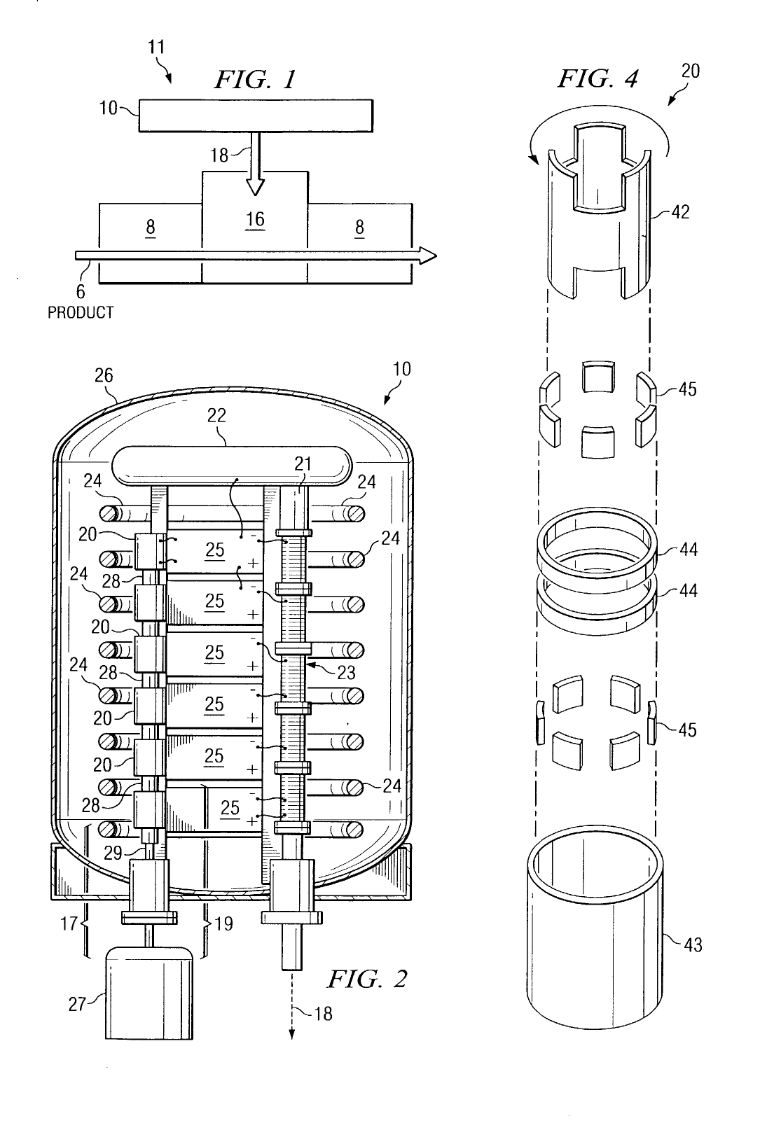

FIG. 1 illustrates a simplified block diagram of one embodiment of a coupled electronic pasteurization system 11. The coupled electronic pasteurization system 11 is related to the electronic pasteurization system disclosed in pending U.S. patent application Ser. No. 09/248,675, entitled Method and System for Electronic Pasteurization, having a filing date of Feb. 11, 1999, which is hereby incorporated by reference as if repeated verbatim immediately hereinafter. In the embodiment illustrated, the improved electronic pasteurization system 11 includes a coupled accelerator 10 and a treatment station 16. As described in greater detail below, the coupled accelerator 10 includes a coupled multiplier supply (CMS) that converts mechanical power to electrical power delivered to certain components of the coupled accelerator 10. Conventional accelerators generally utilize electrical devices, such as transformers, to communicate power to components of the accelerator.

A transport system 8 operates to transport a product 6 through the treatment station 16. While transiting through treatment station 16, product 6 is irradiated by an electron beam 18 produced by the coupled accelerator 10. Within the treatment station 16, the electron beam 18 interacts with the product 6 to affect the desired result, e.g., food pasteurization, water or wastewater purification, chemical decomposition, sterilization of medical instruments and articles, and various others. In a particular embodiment, the product 6 comprises contaminated water that is irradiated by the electron beam 18 to break down, or destroy, certain contaminates in the water, such as bacteria, chemicals, and the like. In another embodiment, the product 6 comprises food products that are irradiated to destroy pathogens, such as bacteria, germs, and the like. In yet another embodiment, the product 6 comprises mail or other packages and the electron beam 18 irradiates the mail to destroy possible pathogens, such as bacteria like anthrax, viruses like small pox, and the like.

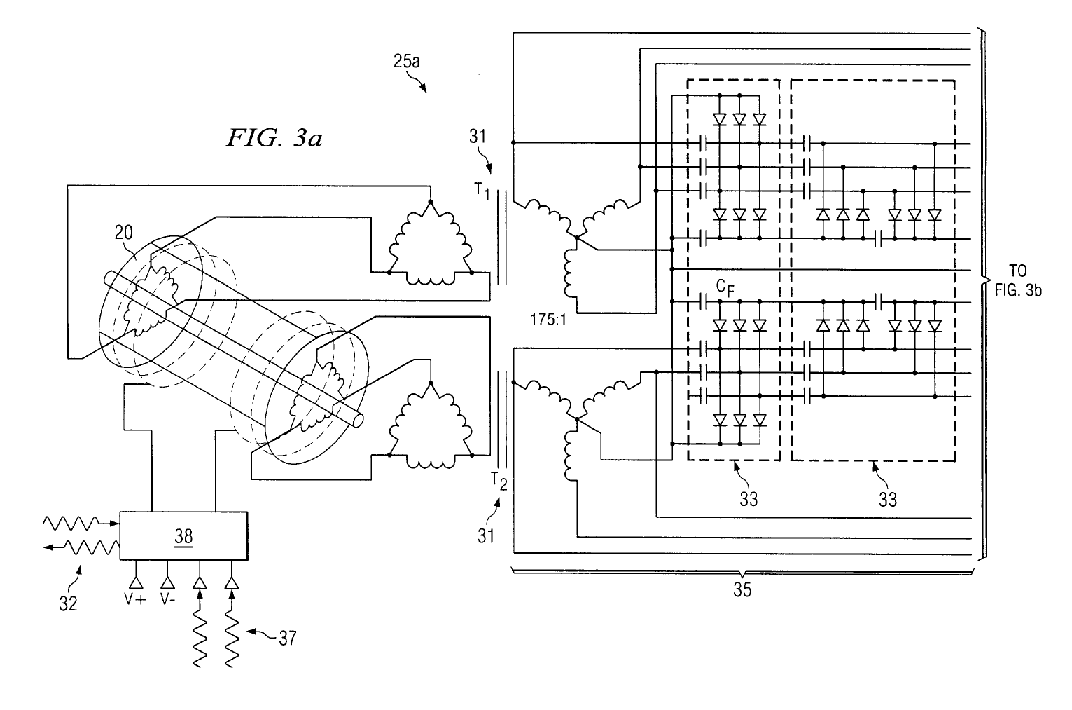

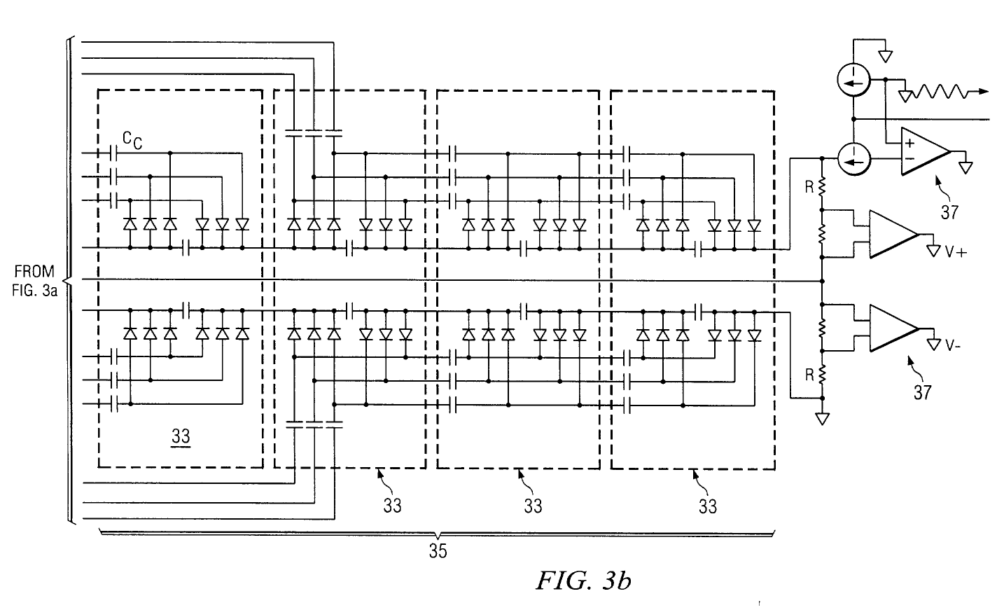

FIG. 2 illustrates a coupled accelerator 10 in accordance with at least one embodiment of the present invention. The coupled accelerator 10 includes a coupled multiplier supply (CMS) 19, a high voltage terminal 22, an electron gun 21, an electron accelerator column 23, an enclosure 26, and an external control computer (not illustrated). The CMS 19 comprises one or more power modules 25 mechanically coupled to a mechanical drive system 17. Each power module 25 converts mechanical energy into electrical energy. In the embodiment illustrated, each power module 25 includes an alternator 20, one or more 3-phase transformers 31 (FIG. 3a), a multi-stage multiplier rectifier circuit 35 (FIGS. 3a and 3b), and a control circuit 37 (FIGS. 3a and 3b). The drive system 17 delivers mechanical power to the power modules 25. In the embodiment illustrated, the drive system 17 includes one or more motors 27 coupled to a rotating insulating shaft 29, which is coupled to each respective power module 25. The drive system 17 may also include one or more flexible insulating (torque) couplings 28 to electrically isolate the power modules 25. In a preferred embodiment, control signals from the external control computer (not illustrated) are transmitted to and from the power modules 25 via fiber optic cables 32 via a modem/data acquisition module 38.

The enclosure 26 serves to contain all of the power modules 25 and load components, e.g., electron gun 21, electron accelerator column 23, etc., within a Faraday cage (not shown) surrounding the modular power supplies 25 so that minimal electrical transient can escape from the power supply assembly in the event of a discharge to ground within the assembly. The enclosure 26 may be a tank or other suitable vessel filled with an insulating liquid, such as mineral oil, or with an insulating gas such as sulfur hexafluoride (SF6), which may be pressurized to increase its dielectric strength. The insulating medium serves to provide high-voltage isolation of the power supply and load components from the ground-potential wall of the vessel. A succession of guard rings 24 may be used to surround the assembly of power modules 25 and loads to present a uniformly graded potential distribution along the insulating gap between guard rings 24 and ground. In a particular embodiment, an insulating gap of approximately 30 cm is sufficient for operation to 1 MV, and a gap of approximately 45 cm is sufficient for operation to 2 MV.

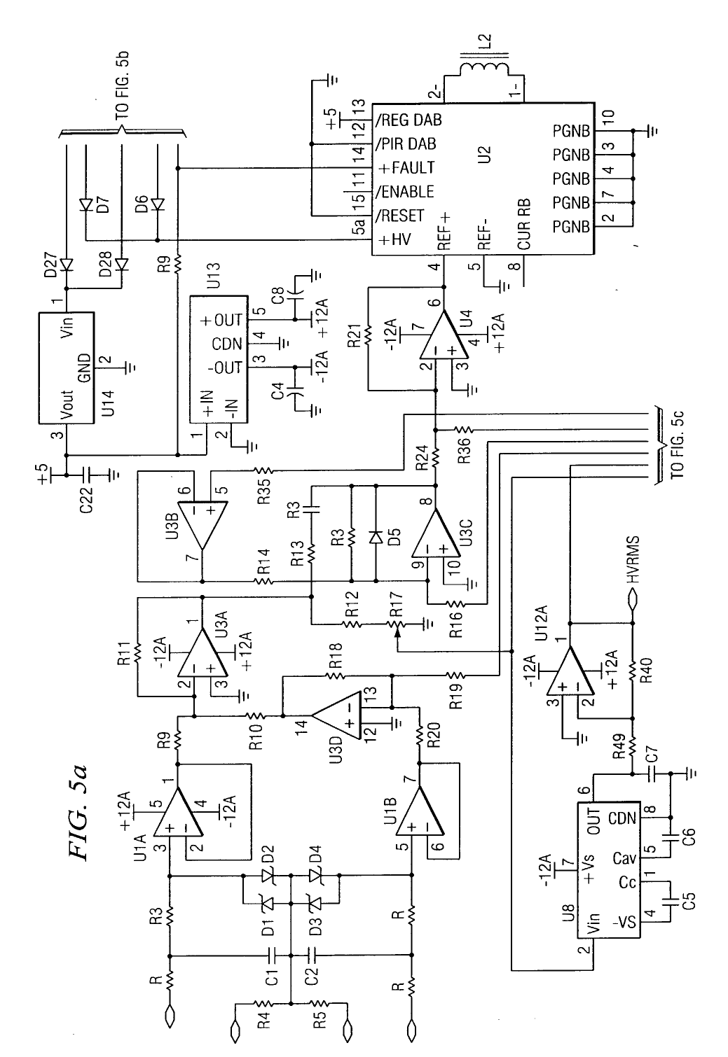

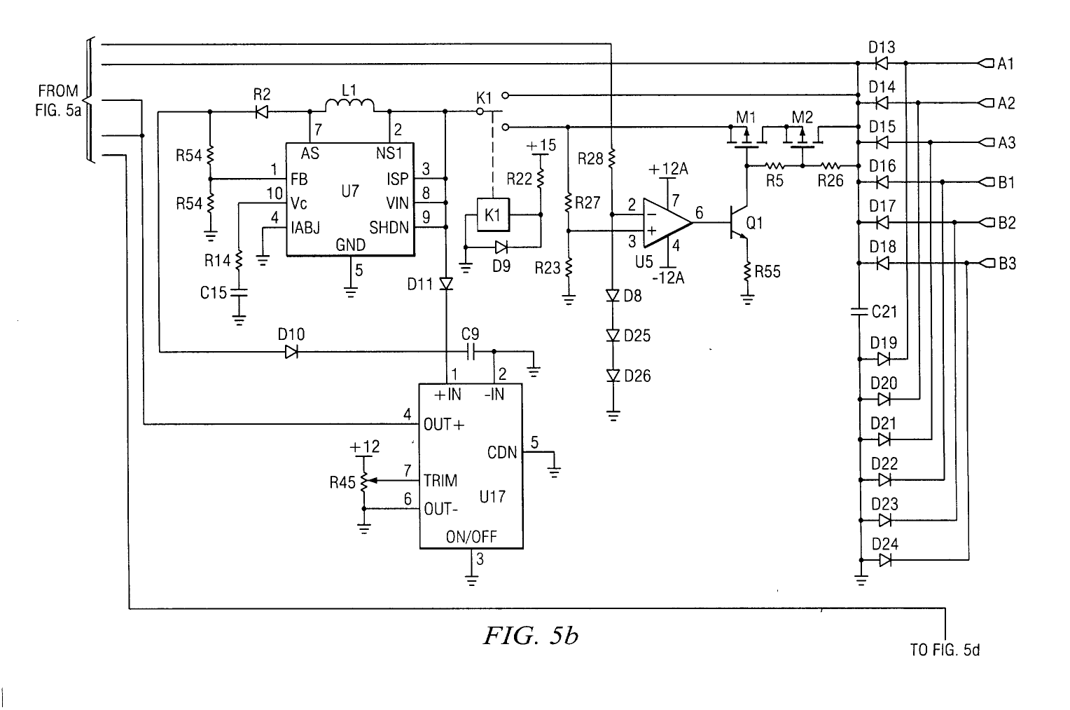

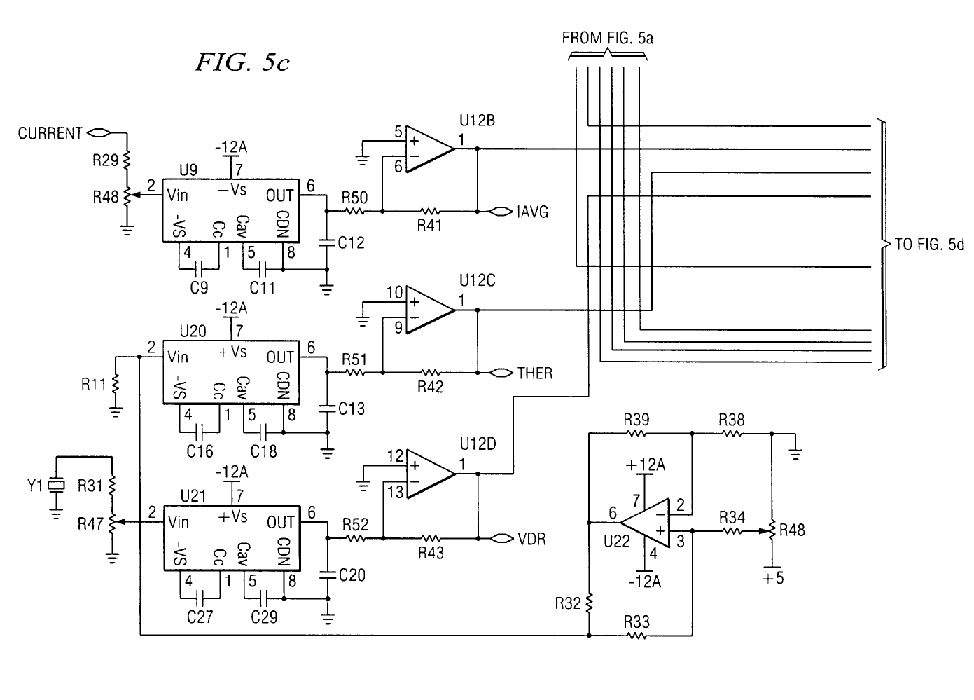

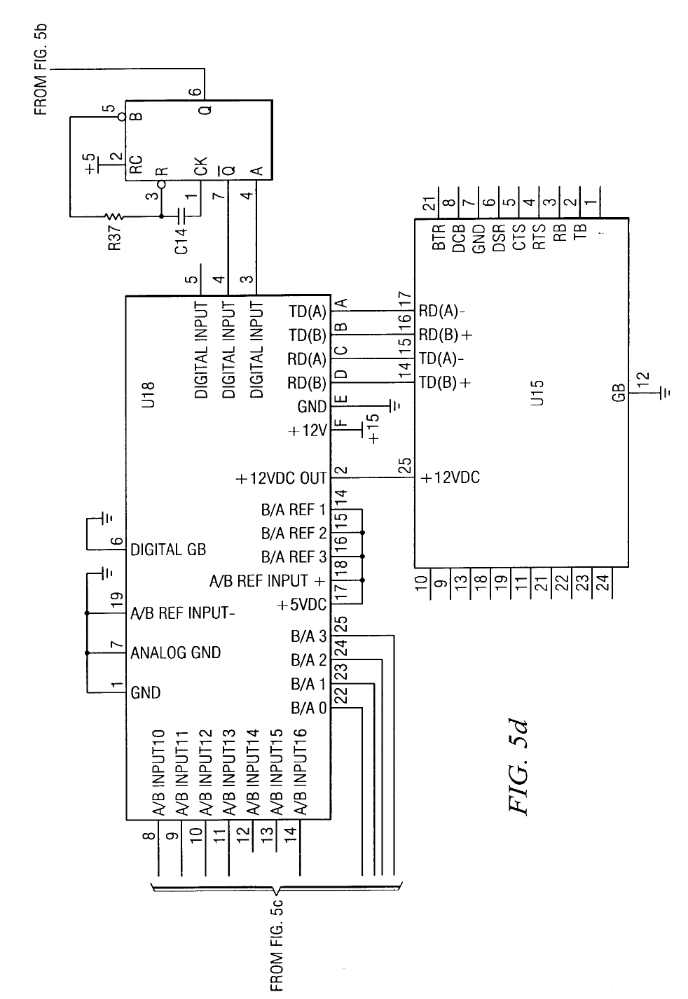

FIG. 3 is an electrical schematic diagram of one embodiment of a power module 25. In this embodiment, the power module 25 includes alternator 20, control circuit 37, two high-voltage (HV) transformers 31, and six full-wave multiplier stages 33. The six full-wave multiplier stages 33 of the detail in FIG. 3 are surrounded by the six dashed-line boxes within the schematic diagram. Control circuit 37 is detailed further in FIG. 5.

The 3-phase pattern of current pulses from the alternator 20 coils is connected to 3-phase transformers 31, typically connected via a delta-wye configuration as illustrated. The transformers 31 are typically wound with a turns ratio N of secondary winding turns to primary winding turns. The 3-phase transformers 31 are used as impedance transformers: when the pulse of rms voltage, Va, and rms current, Ia, is applied to each primary winding, a pulse of rms voltage Vs=NVa, and rms current, Is=Ia/N, is produced in the secondary winding.

As illustrated, the multi-stage multiplier circuit 35 is connected to the 3-phase secondary windings of the transformers 31. The circuit 35 consists of a network of rectifiers and capacitors that rectifies the alternating current of the pulse sequence into a direct-current source of nearly constant vollage, Vp. The multiplier is arranged in a sequence of M stages 33, connected in series. The example shown in FIG. 3 contains M=6 full-wave multiplier stages 33. Each stage 33 is coupled to the next using the coupling capacitors Cc. The a.c. ripple in succeeding stages is filtered using the filter capacitors Cf. The circuit in each stage 33 is typically configured as a full-wave 3-phase multiplier bridge, as seen in FIG. 3. In a multiplier circuit of M full-wave stages 33, connected in series, the output is a d.c. voltage Vₘ, obtained by the equation:

Vₘ=2\sqrt{2}V_{S}=2\sqrt{2}Vₘ=√2 MVs=2√2 MN Va.

Accordingly, the d.c. current Iₘ in a multiplier circuit of M ful-wave stages 33 is obtained by the equation Iₘ=Ia/(2√2 MN), and the lowest frequency component in the a.c. ripple that accompanies the d.c. voltage is fr=2M fa.

FIG. 4 is a schematic diagram of an alternator 20 in accordance with one embodiment of the present invention. Alternator 20 generally consists of 1) one or more pairs of assemblies of stator coils/windings 45, each assembly forming a 3-phase assembly, 2) a rotor 42 consisting of a magnetically permeable element, 3) a field coil 44, and 4) a magnetically permeable case 43. The assembly of these elements within alternator 20 is shown in FIG. 4. The rotor 42 is shaped so that, in each of a succession of rotational positions, its magnetically permeable material couples flux from one of the windings in one stator 45 winding assembly to one of the windings in another stator 45 winding assembly. The field winding 44 creates a magnetic flux which, when each pair of stator 45 windings are thus connected by the rotor's 42 magnetic circuit, connects through that pair of stator 45 windings and returns through the magnetically permeable case of the flux return shell 43. As the shaft (not shown) rotates, the flux threading the coil changes and a pulse of current is excited within each pair of stator 45 windings. The current increases as the shaft rotates into the coupling orientation where the rotor 42 magnetically couples the stators 45, and then decreases as the shaft rotates out of the coupling orientation. In each full revolution, each 3-phase stator 45 winding produces six pulses of current.

In the embodiment illustrated, alternator 20 converts mechanical power from the drive system 17 into electrical power in the 3-phase circuits of its winding assemblies. Accelerator 20 is typically a low-impedance source: it produces electrical power with low voltage Va and high-current Ia in each stator 45 winding. The output power Pa=Vala is controlled by the current in the field coil 44, which produces the magnetic flux that is generating the pulses. The power is approximately proportional to the field coil 45 current, If. The frequency with which the pulse pattern repeats is just six times the revolution frequency, fa, of the alternator shaft (not shown). A typical motor operates at fm=3,600 revolutions per minute=60 Hz. The 3-phase pulse pattern thus has a fundamental frequency fa=360 Hz.

An advantage of the alternator 20 as a power source is that it does not use any brushes that make and break current paths as the rotor 42 turns. For this reason it does not produce any sparks. Such sparks, if produced in routine operation, could dissociate the molecules of any insulating gas, e.g., SF6, and produce corrosion at the metallic surfaces of the electronic components of the coupled accelerator 10.

As an example of convenient choices for the values of the design parameters, each alternator 20 that was used in a first working model produces 8 kW of power, with an rms voltage of approximately 100 V on the 3-phase windings. The pulse pattern has significant harmonic content at higher multiples of fa. The rms amplitude of sinusoidal voltage at the fundamental frequency fa is Va ˜60 V. The transformer (31, FIG. 3) with a turns ratio N=175 would produce a secondary voltage Vs=10,000 V. In the absence of load, the 6-stage full-wave multiplier circuit (35, FIG. 3) would produce a d.c. output voltage Vₘ=120,000 V. The fundamental frequency of ripple is fr=12fa=4,320 Hz. Under full rated load (100 mA), with the values chosen for the capacitors (Cf=0.05 μF, Cc=0.05° F.) the output voltage is 75,000 V. It should be noted that there is a trade-off between the size of the coupling and filter capacitors (larger values give smaller load reduction of Vₘ) and the number of stages to produce the desired total output voltage of the system. The values chosen in this example represent an approximate cost minimum.

FIGS. 5a, 5b, 5c, and 5d illustrate one embodiment of a control circuit 37. FIG. 5a that portion of the control circuit 37 for the control and drive for the field winding. FIG. 5b illustrates the portion of the control circuit 37 for power and startup. FIG. 5c illustrates the portion of the control circuit 37 for the current, vibration, and temperature monitoring, while FIG. 5d illustrates the portion of the control circuit 37 for the data acquisition and optical interface modules.

The output power from each alternator 20 may be controlled by the field strength produced in the alternator 20 by the field winding L2, FIG. 5a. In this embodiment, the control circuit 37 consists of a current source U2, FIG. 5a, that drives current in the field winding L2, a resistive voltage divider, and control electronics that communicate to a master control system. The resistive divider consists of a series network of resistors, of network resistance R, and an output resistor r connected to the case ground of the power module 25 as shown in FIG. 3b.

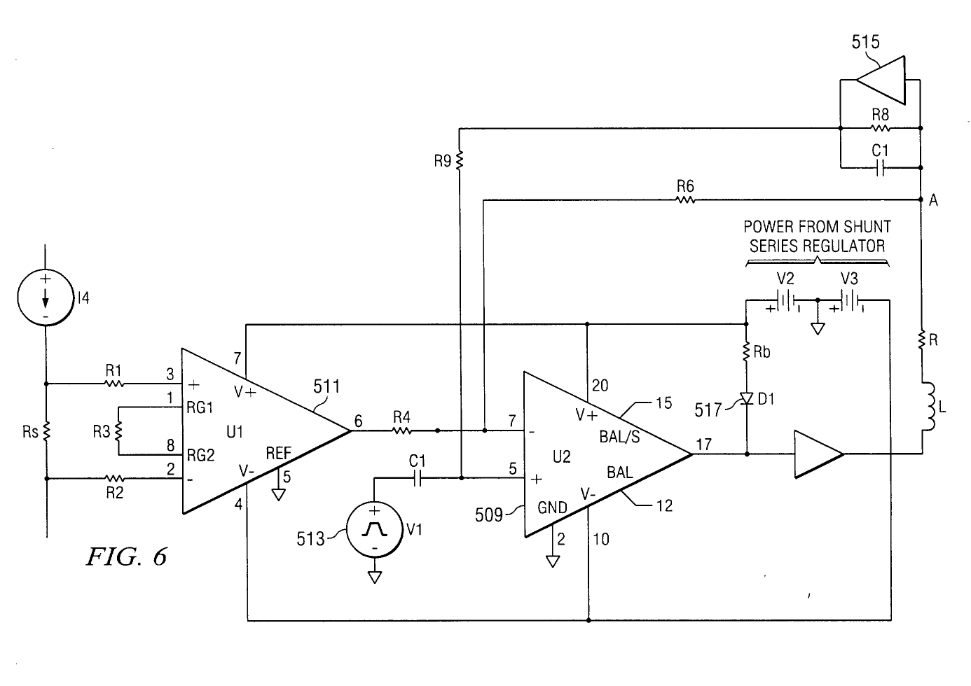

The control circuit 37 preferably includes a data acquisition and optical interface shown in FIG. 5d, vibration monitor Y1, FIG. 5c, and heat sensors R1, also in FIG. 5c, a current monitor (FIG. 6) and voltage monitor, startup power and operating power circuits. The data acquisition (DAQ) module U18 in FIG. 5d, and a fiber optic interface module U15 shown in FIG. 5d are provided in each control circuit 37. In the embodiment illustrated, the DAQ module U18 consists of seven analog-to-digital (A/D) converters and four digital-to-analog (D/A) channels. The A/D channels monitor output high voltages, current, in/out current difference, temperature, vibration, and internal signals relative to the operation of the controller. The D/A channels provide the command voltage to which the module output is to be regulated, diagnostic signals, and safety control signals. The fiber optic interface module U15 routes the inputs and outputs from the data acquisition module U18 via an optical cable (not shown). This optical cable is daisy chained to all the other control circuits in the system, including a remote computer system (not shown) via a RS485 serial port.

In a preferred embodiment, the d.c. power to operate all control circuitry is provided by a startup battery (not shown), and then by an operating power circuit. When the alternator 20 is first rotated from a stop position, the alternator 20 generates a small voltage at its terminals due to the remnant magnetic fields in the steel flux return (43, FIG. 4). Since this voltage is not yet sufficient to power the control electronics, power is initially derived from a 12 V battery. The operating power supply rectifies the a.c. voltage from the alternator terminals and produces the regulated voltages required for the control electronics. As output power is increased, the terminal voltage from the alternator 20 increases, and when the output power reaches approximately 18 V, the voltage source for the operating power supply is switched from the battery to the rectified terminal voltage. In this normal operating mode, the battery is charged from the rectified output of the alternator 20.

The alternator 20 output voltage is proportional to the current supplied to the field coil (44, FIG. 4). At startup, no field current can exist. It has been determined that the weak magnetic field due to local magnetization of the surrounding metal of the alternator 20 can provide the necessary startup power. The controller startup circuit has various components: dc-dc boost converters U17 and U7 in FIG. 5b, a series regulator M1, M2, U5, and Q1 in FIG. 5b, and a 12-volt battery (not shown). The initial voltages produced by the alternator at startup are too small for the control circuits to use (˜2-3 V). The dc-dc boost converter U7 is therefore utilized during startup. This converter takes the low alternator output voltage (˜2-3 V) and steps it up to 9 V. This stepped-up voltage drives a second dc-dc boost converter U17 that powers the circuits necessary to provide field current to the alternator 20, and also powers the interface and control circuitry. Once field current is initialized, converter U17 is switched out, and the main series regulator provides power directly to converter U7. Converter U7 provides power to all electronics until the alternator output voltage is of sufficient amplitude (˜22 V) to power the pulse width modulator (pwm). Converter U7 then only powers control and monitor electronics.

A pulse width modulator (pwm) U2, FIG. 5a, provides current for the field windings. The pwm is the most efficient method for providing current to the field windings L2, FIG. 5a, of the alternator 20. The output of the pwm U2 is a stream of pulses of fixed period. The duty cycle of the pulse stream adjusts under feedback control to meet a specified current output. In the preferred embodiment, the control input to the pwm U2 is a voltage in the range of 0 to 10 volts. A control voltage of 1 V generally produces an output current of 1 A.

A voltage divider network (resistors R, r in FIG. 3) may be placed between the positive high voltage output and the common midpoint in each power module. A second voltage divider network is placed between the negative high voltage output and the common midpoint. The voltage across each output resistor r is Vd=(r/R)Vm. For a typical choice R/r=4000, each divider output at full excitation would be Vd=8 V. The divider outputs provide a convenient measure of the output voltage (voltage monitoring) of the power module 25.

In the embodiment of divider networks, the two divider outputs are sampled and summed in the interface. The command voltage from the interface is subtracted from the summed voltage and integrated to provide an error voltage. This error voltage is applied to the control voltage input of a pulse width modulator that generates the field current for the alternator. This is the control loop by which the high voltage output of each control module is regulated to a desired value that has been communicated to the interface through its fiber optic interface. The steady state condition approach forces the integral gain towards infinity, stabilizing the high voltage outputs at the desired voltage.

Monitor circuits, an embodiment of which is illustrated in FIG. 6, are preferably inserted in the positive and negative high voltage outputs of each power module 25, as seen in FIG. 3. It is desirable to monitor both the current passing through each module and the difference between the currents passing through successive modules. In many applications, the outputs from successive modules are connected to a load, for example the electrodes of the accelerator column. In such applications, the difference current would provide a sensitive measure of the presence of either corona discharge from elements in the power module, or interception current on the electrodes connected to it. A summing amplifier is used to take the difference of these currents.

In the embodiment of the circuit for the current monitoring sensor shown in FIG. 6, the current sensor is an insertion device which generates the voltages needed for the operation of its active components from a small voltage drop across the insertion point. In FIG. 6, the sense resistor Rs (Rs=1 Ω for 0-100 mA response) develops a voltage proportional to the current output of the power module. An instrumentation amplifier 511 amplifies this voltage signal with a gain of 100. Its output, Vi=100 Irs, voltage is fed to the negative (−) input of the comparator 509.

In a specific embodiment, a train of sawtooth pulses is generated by a saw-tooth generator 513 (frequency 20 kHz, amplitude 11 V peak-to-peak), and is a.c.-coupled to the positive (+) input of the comparator 509. The comparator 509 output is in its saturated on state whenever the signal voltage from the instrumentation amplifier 511 is greater than that of the sawtooth 513. The output of the comparator 509 is buffered and applied through an LR filter to summing junction A, together with a current through resistor R6 from the buffered Vi. Error amplifier 515 feeds back on the + input to maintain null at the summing junction A. Providing that the period of the sawtooth is small compared to the L/R relaxation time of the filter, the width of each saturated pulse out of the comparator is nearly proportional to the current I to be measured.

The buffered output of the comparator is applied to a light-emitting diode (LED) 517. The diode 517 thus emits a 20 kHz train of pulses, in which the pulse width is proportional to current I from the power module. The light output is conveyed to the control circuit via a fiber-optic cable.

An advantage of the above embodiment is that the response is independent of variations in the amplitude of light emitted by the LED 517, therefore making the disclosed technique superior to amplitude-modulated transduction. In addition, information is transmitted at fixed frequency so that the response to small signals cannot take the transmitted pulse train out of the range of response for a receiving circuit, making the disclosed technique superior to voltage-to-frequency transduction. The method also eliminates the need to send a return signal back from receiver to transmitter to achieve linearization.

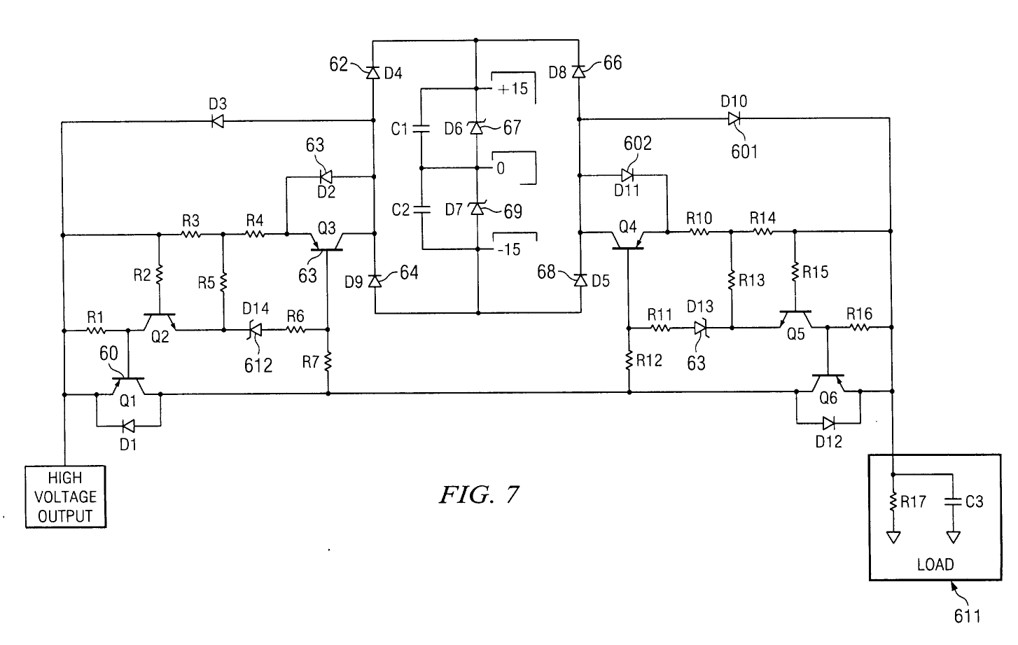

A receiving circuit, which may be located in the control module, reverses the above sequence, and produces a voltage that is proportional to the pulse width in the transmitted light signal, and therefore proportional to the control module current I. A series shunt regulator, seen in FIG. 6 and shown in greater detail in FIG. 7, provides the ±15 V d.c. power needed for operation of this embodiment of the current monitor. The regulator is inserted in series with the output of the power module. The regulator must provide for operation of the current monitor during normal operation of the power module, when >1 mA is being sourced by the current module, and also for operation during startup and idling (I<1 mA), when the regulator must generate the necessary supply voltages from a battery source.

The shunt/series regulator provides two paths for the current to flow, as seen in FIG. 7. During low current operation, Q1 (60) is off and all the current is diverted into Q3 (61). Diodes D4 (62), D5 (64), D8 (66), D9 (68) form a full wave bridge around zener diodes D6 (67), D7 (69). The bridge forces the current always to flow so that voltages developed across the two zener diodes are always of the proper polarity. Bridge current exits thru D10 (601), D11 (602) into multiplier load R17 (611). The zener diode requires 1 to 30 mA. As the supply current I from the power module increases, Zener diode D14 (612) turns on and shunts additional current through Q1 (60), so that it does not pass through the regulator.

It is also desirable to measure the difference between the currents sourced by one power module and sinked by the next, in order to measure intermediate load currents that could signal problems in the overall supply and its load. For this purpose, two such current monitors are generally connected in series as shown in FIG. 3. The output of one of the monitors is conveyed via fiber-optic cable to the control circuit as discussed above. The outputs of both instrumentation amplifiers can be connected to a difference amplifier, and the voltage corresponding to the difference current is pulse-width-encoded in the same manner as in FIG. 6, but the value of L/R time constant is increased by a factor of 1,000 so that the range for measurement of difference current is 0-100 μA (1,000 times more sensitive than the measurement of I). The light output is conveyed via optical fiber to a second receiver in the control module.

In this way, at each power module 25 output both the current passed to the next power module 25 and the current that is delivered to an intermediate load connected to that module 25 are measured. In an application where the coupled multiplier supply is used to power an accelerator 10, this provides an important diagnostic, in which the current to the intermediate load consists of interception current (electron beam 18 lost to an electrode along the accelerator column 23) and corona discharge (current conveyed by high-voltage breakdown to ground). Both currents are undesirable. The series of diagnostic current measurements would make it possible to localize where in the supply any such problem developed.

The control circuit 37 can monitor the temperature using a thermistor that is mounted to the case of the alternator. A thermistor is a device whose resistance is a strong function of temperature. The voltage developed across the thermistor is digitized in the interface. The control computer uses a look up table to determine actual temperature.

Vibration can also be measured by a transducer that is mounted on the alternator 20 case. A piezoelectric crystal transducer produces a voltage at its output terminals that is proportional to pressure along its axis. The transducer voltage is fed to a high-impedance rms converter, and the rms signal is digitized in the interface. The temperature and vibration measurements enable monitoring of each alternator for conditions of overheating or failure of mechanical bearings that could jeopardize the function of the system.

In certain embodiments, alternators 20 of the succession of power modules 25 are preferably connected in series via insulated flexible torque couplings/isolation hubs 28, as was briefly discussed with reference to FIG. 2. In a particular embodiment, the succession of alternators 20 is connected in two such series strings, with alternators 20 of succeeding power modules 25 installed alternately on the two strings. In this way, the overall assembly length is minimized, and the voltage between two successive alternators 20 in each string is 2 Vm. With the choice of Vm=65 kV in the example previously discussed, successive alternators 20 must operate at a voltage difference of 130 kV on their cases.

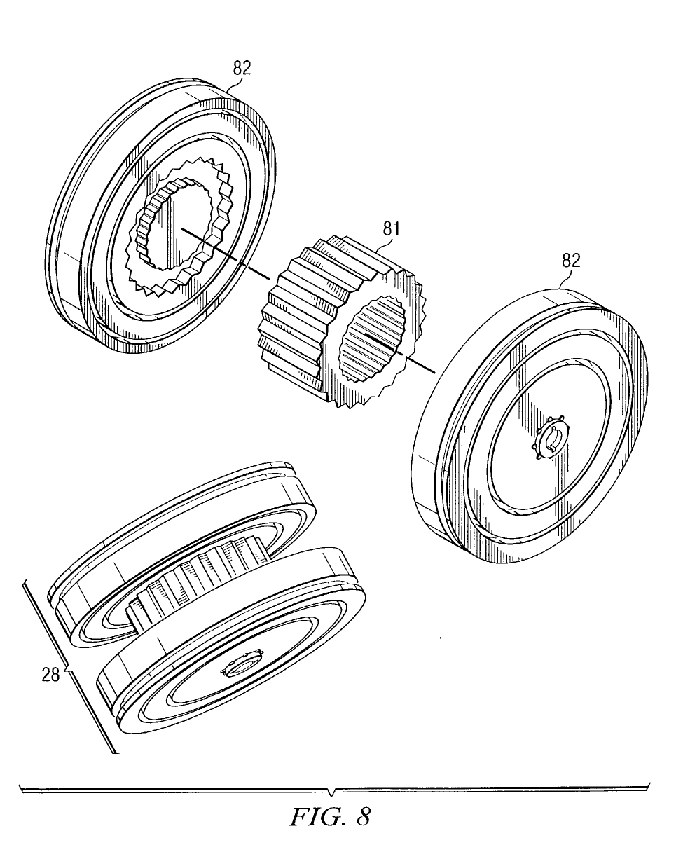

The torque coupling/isolation hub 28, illustrated in FIG. 8, can be used among the alternators 20 of each string, to drive the string from a motor 27 mounted outside the enclosure 26. In one embodiment, successive alternators 20 are coupled using a standard flexible coupling 81 composed of an electrically insulating plastic material such as HYTRIL™. This flexible coupling 81 is captured between two structural hubs 82 composed of a rigid, high-strength, electrically insulating plastic material such as G-10. The HYTRIL™ coupling element 81 is electrically insulating, but mechanically durable and flexible. The G-10 plastic hubs 82 may be fabricated with a succession of surface grooves that provide multiple blockages for surface breakdown currents. One example of a grooved G-10 plastic hub 82 is shown in FIG. 8. Although HYTRIL and G-10 plastics are cited in the example, other suitable plastics materials known to those of skill in the art can be substituted for the HYTRIL™ and G-10.

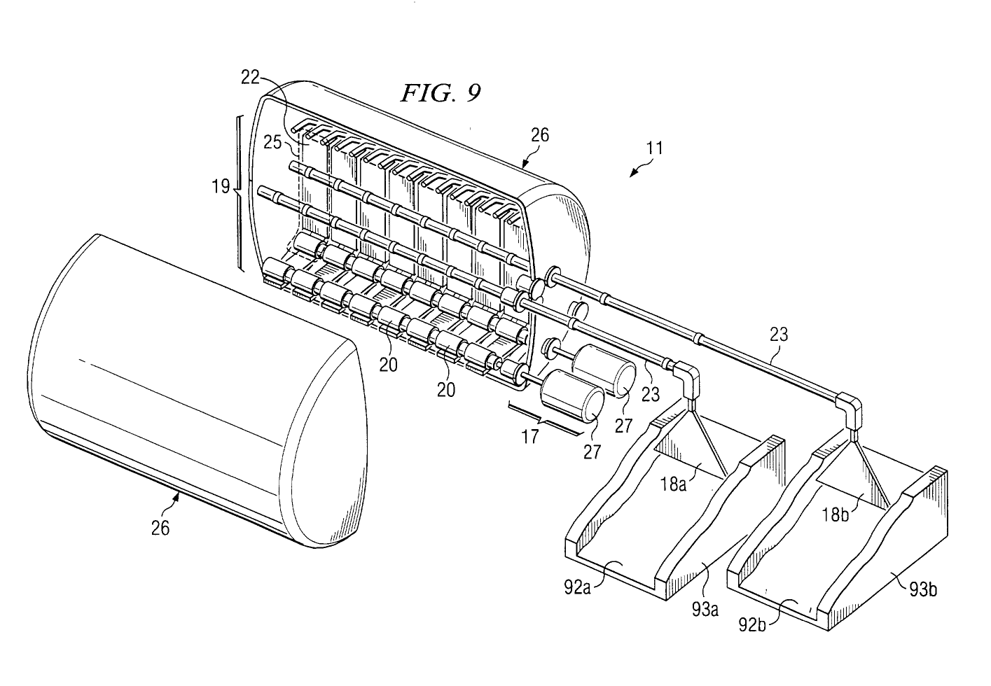

Each string of alternators 20 is driven by drive system 17 that generally includes motors 27 external to the enclosure vessel 26, as shown in FIG. 2, and FIG. 9. FIG. 9 is a perspective cutaway view of the coupled multiplier supply 19 integrated with two electron accelerators as load for a coupled pasteurization system 11 in accordance with the present invention. Each motor 27 is powered through a conventional starter circuit (not shown), which enables automatic shut-down in the event of over-current, and also external triggering of shut-down if the optical fiber interface connection be lost, the control computer malfunction, or the control circuit of any power module 25 fail to respond or read values that indicate problems.

The coupled multiplier supply 19 seen in the simplified diagram of FIG. 9 includes alternator 20 stacks, and high voltage modules 22. The transformers and control circuitry for the coupled multiplier supply 19 are not shown in FIG. 9. The coupled multiplier supply 19 permits a variety of load connection possibilities. Its ability to provide multiple stages, with independent voltage control and current monitoring on each stage, is an advantage for some applications. The load illustrated in the example of FIG. 9is an electron accelerator, to which one or more accelerator columns 23 are connected to the succession of voltages 22 from the coupled multiplier supply 19, and used to accelerate multiple independent beams of electrons 18a, 18b to MV energies. FIG. 9 illustrates one example configuration of a 1 MV coupled-multiplier supply 19 powering two electron beams 18a and 18b treating two liquid streams 92a and 92b flowing over two weirs 93a and 93b.

Over a decade of laboratory testing and water chemistry studies utilizing electron-beam technology have demonstrated the efficacy of an electron beam in dissociating organic contaminants in water. Still, the technology has not proven to be cost-effective on an industrial scale due to the large initial capital expenditures as well as operational costs, as previously discussed. However, an embodiment of the present invention can be utilized for treating liquid streams 92a, 92b such as water or wastewater in a cost-effective manner. The coupled multiplier supply 19 of the electronic pasteurization system 11 disclosed herein is a completely self-contained, high power electron accelerator that can produce 100 kW of beam power at 1 MV, supports multiple independent electron beams 18a, 18b, at a capital cost of about one-third that of conventional industrial electron beam accelerators.

An example of a water treatment application for which the present invention is well suited is as a treatment system for condensate streams containing methyl terbutyl ether (MTBE), an oxidation-enhancing additive in gasoline. MTBE is highly resistant to removal by conventional bio-digestion, or by mixed beds and membrane systems, particularly because a number of the secondary products, e.g., formaldehyde, acetone, formed in these bio-digestive processes exhibit greater toxicity than MTBE. The digestion of aromatic hydrocarbons such as MTBE typically requires a sequence of both oxidation and reduction reactions. Electron-beam treatment creates ionization and excitation of the water and MTBE molecules. This excitation/ionization produces large concentrations of short-lived, highly reactive radicals, e.g., H., OH., and aqueous electrons e⁻_{aq}, which produce both oxidation and reduction reactions within the liquid stream, thus driving digestion through several intermediaries to non-toxic end products.

Various studies, i.e., Cooper et al., Zele et al., Bolton et al., Nickelsen et al., and Waite et al., employing electron-beam treatment for MTBE and other aromatics such as benzene, phenol and other halogenated aromatics, e.g., n-chlorobiphenols and hexacholorbenene have demonstrated the effectiveness of the technology. For example, electron-beam treatment has been shown to produce dramatically superior results and cost-effectiveness over other advanced oxidation technologies such as UV/H2O2 and UV/TiO2 in the decay of phenol. However, the current processing costs of a conventional full-scale electron-beam treatment system (75 kW power for the electron beam) have been modeled by Kurucz et al. to be on the order of $5.00/1000 gallons for a 4 kiloGray (kGy) delivered dose. The same modeling projections for the coupled multiplier supply electronic pasteurization method (100 kW power for the electron beam) as disclosed herein indicate the treatment costs to be on the order of $1.40/1000 gallons for the same delivered dose.

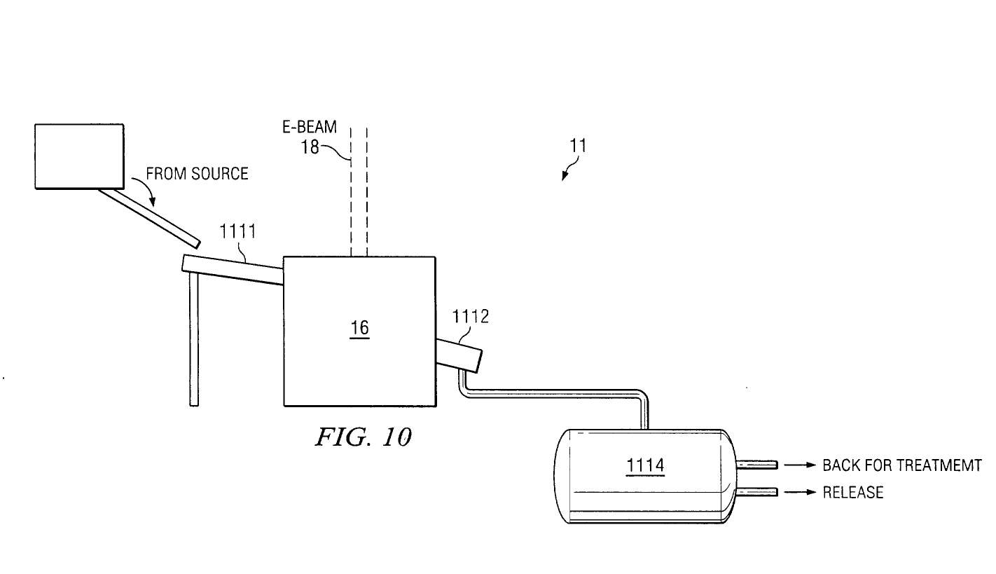

The improved pasteurization system as disclosed herein is a cost-effective means of treating of a chemical compound in a waste stream. When the target is a chemical compound, the electron beam operates as a catalyst with the chemical compound, altering its composition. This concept is illustrated in FIG. 10, which illustrates a simplified diagram of improved pasteurization system 11 being used to treat a chemical compound waste stream 1111 having a flow rate greater than 1 cubic foot per minute with an electron beam 18 in a treatment station 16. In a particular embodiment, electron beam 18 has an energy greater than 2 Mv.

After electron beam 18 treatment, the treated waste stream 1112 may be routed to a holding tank 1114. The treated waste stream 1112 contained in holding tank 1114 may be sampled for analysis to determine if the desired result has been achieved. If analysis of treated waste stream 1112 indicates suitable results, the treated waste stream 1112 may be released. If analysis reveals further electron beam 18 treatment is in order, the treated waste stream 1112 may be routed back through the treatment station 16 for additional treatment. Although only one electron beam 18 is shown in the example of FIG. 10, because the present invention is capable of producing a plurality of electron beams, multiple treatment stations or various types of treatment loops may be employed to treat waste stream 1111.

In addition to utilizing the present invention to treat liquid waste streams, the method is also suited to treatment of a gaseous waste stream. This could be accomplished by use of the improved electronic pasteurization system 11 with, for example, a flue, gas stack, or treatment loop transporting a continuous flow of a gaseous waste product for treatment before release to the environment.

An advantage of at least one embodiment is that more power modules can be added to increase the voltage. The cost and size grow linearly. As the current needed in an application increases, one simply increases the capacity of the alternators 20 and the rectifier diodes proportionately. This linear relationship reduces the costs associated with fabricating a coupled accelerator and the improved electronic pasteurization system 11.

By contrast, conventional d.c. high voltage supplies generally operate either by coupling energy capacitively through a succession of series stages, e.g., a Cockroft-Walton accelerator or dynamitron, or by coupling energy magnetically through a succession of transformers, e.g., insulating-core transformers. In either case, there is a “pyramid effect” because the energy that is eventually delivered to the high-voltage terminal must be pushed through all of the stages. The lowest stage must deliver the power needed to maintain its voltage as well as the power needed for all succeeding stages. The result of this pyramid is that the quantity of components, and their size and cost scale roughly quadratically with the terminal voltage and/or the terminal current. The present invention breaks the back of this quadratic dependence.

An advantage of at least one embodiment of the improved pasteurization system 11 is the CMS 19 connected to the power modules 25. Although the examples presented herein present electric motors 27 as part of the drive system 17, different types of drive systems, e.g., transverse belt drives and others known in the art, may be employed in the drive system 17. The use of a common drive system has the advantage of generating electrical power at high voltage that is controlled by means of field currents to the alternators 20 of each power module 25. The phasing of the alternators 20 of successive power modules 25 can provide suppression of a.c. ripple and pulsed load currents with a modest amount of filter capacitance.

Another advantage of at least one embodiment is that the linear dependence of cost and the size of the supply on the output voltage and current required can be reduced. Furthermore, in electron beam applications, the isolation of the entire system from any potential to create electrical transients on external power lines and ground conductors in the event of an arc to ground may be advantageous. These features furthermore facilitate the operation of multiple independent electron beams within a single power supply and enclosure, and to maintain control of output voltage even with operation of pulsed current from accelerator loads.

In some embodiments, ripple is strongly suppressed by the 11-stage multiplier in each power module, so that the fundamental frequency of ripple is fr=3,960 Hz. Ripple can be further suppressed by step-phasing the orientation of the rotors on the alternators of successive power modules. For example, if the 16 modules shown in FIG. 9 are connected in series, an output voltage Vout=16 Vm=1,000,000 V is produced. The rotors of the 16 alternators can be positioned at successive phase advances of 360°/16=22.5° in their interconnection along the drive shaft. The a.c. ripple from successive modules will then have this same distribution of phases, with the result that the lowest frequency of ripple in the series voltage Vout will be 16fr=63,360 Hz. This increase in ripple frequency has the double benefit of reducing the phase width of the individual pulses that produces ripple, and reducing the capacitance needed for a given attenuation factor. It is then practical to provide very low-ripple output, even at very high voltage and high power, with modest cost in filter components.

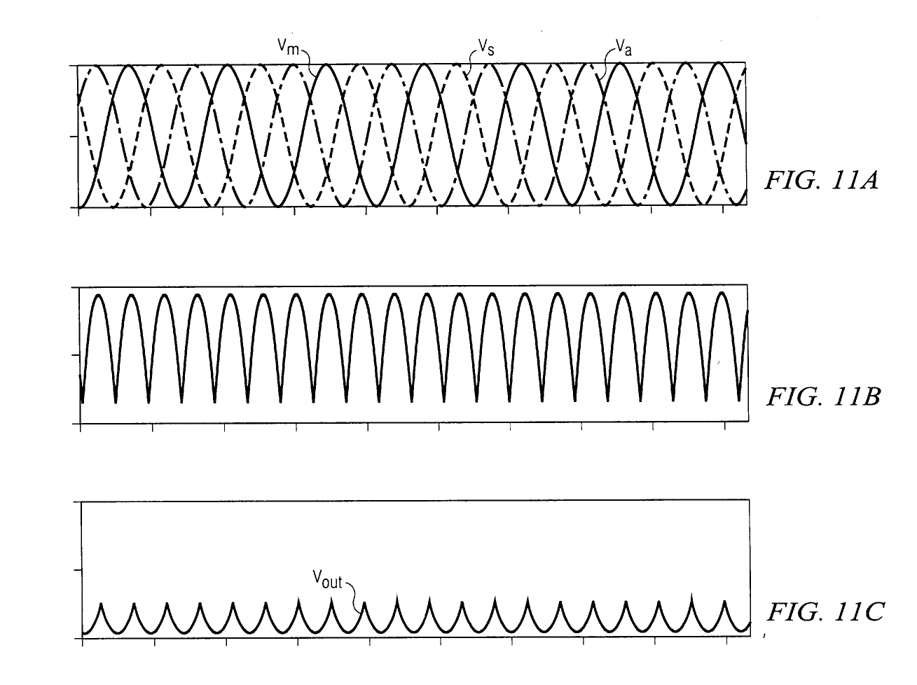

The waveforms of the voltages Va, Vs, Vm, and Vout showing the response of a power module under full load current (100 mA) are shown in FIGS. 11A, 11B, and 11C. FIG. 11A is a graph of the waveforms for Va, Vs, and Vm, while the graph of FIG. 11B indicates these waveforms after full-wave rectification. FIG. 11C indicates the Vout (for the negative half) for the power module under full load current conditions. The time between successive charging cycles with staggered phases of alternators is T=1/32fm. A benefit of this reduction of time between charging cycles is to make it feasible to control load voltage even during pulsed beam operation of accelerator loads. As each pulse of charge is emitted from a cathode in an accelerator 10 that is powered by the coupled multiplier supply 19, the time before charging current is available to recharge the effective output capacitance is reduced by the factor 16 from its value in a conventional rectifier.

In embodiments in which the load(s) are contained within the enclosure 26 with the supply 19, no high voltage need be transmitted outside of the enclosure vessel 26. This provides the advantage that the only power that enters the enclosure 26 is mechanical drive system 17; the only power that leaves the enclosure 26 is in an electron beam 18, 18a, 18b. In the event that an arc were to develop within the enclosure, for example from one of the power modules 25 to ground or from an accelerating column 23 to ground, there is no means by which the arc could produce an electrical transient on the a.c. power lines that deliver power to the system, or on the ground conductors that may be common within those in an industrial plant. The arc is confined within a Faraday cage, with no electrical connections entering or leaving it.

In addition, it is typically not feasible to monitor the pattern of currents flowing up the multiple stages of conventional d.c. high-voltage supplies. In the event that corona occurs or interception currents within intermediate stages of a load, it is therefore difficult to trace the origin of the problem. The coupled multiplier supply 19 allows a self-contained current monitor to be inserted where desired in the series connection of stages 33, which is self-powered by introducing a small series voltage drop in the series connection. The current monitor output is encoded as a pulse train of light pulses and delivered through high voltage isolation to the neighboring control circuit. This self-contained, self-powered, optically coupled monitor allows the improved pasteurization system 11 to be diagnosed and monitored.

Although the present invention and its advantages have been described in detail, it should be understood that various changes, substitutions, and alterations can be made therein without departing from the spirit and scope of the present invention as defined by the appended claims.

What is claimed is:

1. An improved pasteurization system comprising:
a coupled accelerator operable to produce at least one electron beam, the coupled accelerator comprising:
at least one accelerator column;
a coupled multiplier supply having a mechanical drive system and at least one power module, wherein the at least one power module operates to supply power, in part, to the accelerator column; and
at least one electron gun; and
at least one treatment station in communication with the coupled accelerator, wherein the treatment station operates to receive the a least one electron beam and irradiate a target with the at least one electron beam.

2. The improved pasteurization system of claim 1, wherein the mechanical drive system is rotationally coupled to the at least one power module using at least one drive shaft.

3. The improved pasteurization system of claim 1, wherein the power modules are serially coupled together on a common drive mechanism.

4. The improved pasteurization system of claim 1, wherein the mechanical drive system is coupled to the at least one power module using at least one flexible belt.

5. The improved pasteurization system of claim 1, wherein the mechanical drive system includes at least one flexible insulating coupling.

6. The improved pasteurization system of claim 1, wherein the coupled accelerator operates to produce a plurality of electron beams.

7. The improved pasteurization system of claim 1, further including a beam transport system operable to communicate at least one electron beam to a treatment station.

8. The improved pasteurization system of claim 1, wherein the treatment station includes a multilayer shielding system comprising a first layer operable to absorb substantially all electrons and produce low level X-ray radiation, and a second layer disposed outwardly from the first layer, wherein the second layer operates to absorb substantially all of the low level X-ray radiation.

9. The improved pasteurization system of claim 1, wherein at least one power module includes a control circuit.

10. The improved pasteurization system of claim 9, wherein the control circuit is operable to monitor the current and voltage of the power module.

11. The improved pasteurization system of claim 9, wherein the control circuit communicates with an external control computer using an optical interface.

12. The improved pasteurization system of claim 1, wherein the target comprises a liquid waste stream.

13. The improved pasteurization system of claim 1, wherein the target comprises a gaseous waste stream.

14. The improved pasteurization system of claim 1, wherein the target comprises a chemical compound and the electron beam operates as a catalyst with the chemical compound.

15. The improved pasteurization system of claim 1, wherein the treatment station includes a transport system operable to transport the target through the treatment station.

16. The improved pasteurization system of claim 1, wherein each power module comprises:
at least one alternator;
at least one3-phase transformer;
at least one multi-stage multiplier circuit; and
a control circuit.

17. A method for irradiating a target with at least one electron beam comprising:
providing a source of mechanical power;
generating electrical power from at least one power module by mechanically coupling each power module to the source of mechanical power;
supplying the electrical power to an accelerator column to generate a voltage differential within the accelerator column;
accelerating at least one stream of electrons within the accelerator column to produce the at least one electron beam; and
irradiating the target with the at least one electron beam.

18. The method of claim 17, wherein the source of mechanical power comprises at least one motor.

19. The method of claim 17, wherein the at least one power module comprises:
at least one alternator; and
a control circuit.

20. The method of claim 17, wherein the target comprises a liquid waste stream.

21. The method of claim 17, wherein the target comprises a gaseous waste stream.

22. The method of claim 17, wherein the target comprises a chemical compound and the electron beam operates as a catalyst with the chemical compound.

23. The method of claim 17, wherein each electron beam has an energy greater than 2 MV.

24. The method of claim 17, wherein each power module includes a control circuit operable to control the current and voltage produced by the power module.

25. The method of claim 24, wherein the control circuit also operates to monitor one or more data sensors taken from a group consisting of temperature, vibration, battery, voltage, current, start-up power and operating power circuits.
